(19) United States
(12) Patent Application Publication
(10) Pub. No.: US 2024/0404639 A1
(43) Pub. Date: Dec. 5, 2024

(54) Method and device for analyzing a dataset

(71) Applicant: Roche Molecular Systems, Inc., Pleasanton, CA (US)

(72) Inventor: Rolf Knobel, Rotkreuz (CH)

(21) Appl. No.: 18/803,361

(22) Filed: Aug. 13, 2024

Related U.S. Application Data
(63) Continuation of application No. 16/036,711, filed on Jul. 16, 2018.

(30) Foreign Application Priority Data
Jul. 17, 2017 (EP) 17181726.5

(51) Int. Cl. G16B 40/10; G06F 16/28; G16B 40/00; G16B 40/20

(52) U.S. Cl. CPC G16B 40/10; G06F 16/287; G16B 40/00; G16B 40/20

(57) ABSTRACT

The present disclosure is concerned with data evaluation tools, methods for analyzing a dataset, and a computer readable medium comprising a computer program code that when run on a data processing device carries out the method of the disclosure as well as a device for carrying out the method of the disclosure. The methods and devices disclosed herein are used in analytical systems that analyze biological samples.

### CROSS-REFERENCE TO RELATED APPLICATIONS

The present application claims the benefit of priority under 35 U.S.C. § 119 (a) of EP 17181726.5, filed Jul. 17, 2017, the disclosure of which is incorporated herein by reference in its entirety.

### FIELD OF THE DISCLOSURE

The present disclosure is concerned with data evaluation tools and a device for carrying out the methods described herein.

### BACKGROUND OF THE DISCLOSURE

Research and/or diagnostic applications may have unequal sensitivity and specificity expectations. For certain applications, a high sensitivity, i.e., a low false negative rate, is required, whereas for other applications a high specificity, i.e., few false positives, is needed. For examples, in blood screening applications high sensitivity is often regarded as key feature while for detection of certain mutation(s) specificity may be more important.

In particular, data sets derived from highly sensitive biological assays such as polymerase chain reaction (PCR), Flow Cytometry, nucleic acid hybridization, immunohistology and imaging often contain varying signals of positives and negatives and therefore show an overlapping signal distribution (sometimes also called rain). Especially when working in the low concentration range, e.g., less than 1% of partitions called positive, a false positive or false negative partition call can have a considerable impact by increasing or decreasing the reported concentration considerably above or below a threshold indicating presence. This may have a strong impact on the outcome and thus may be of harm. For example, a false medical report based on a false positive signal could be issued, indicating the presence of a certain DNA/mutation (which in reality is not present) and potentially affecting further treatment of the patient.

One of the most sensitive assays to detect an analyte of interest and, in particular, a certain nucleic acid, is digital PCR. In a digital PCR (dPCR) assay the sample is separated into a large number of partitions (aliquots) and the PCR reaction is carried out in each partition individually. This separation allows a more reliable collection and sensitive measurement of nucleic acid amounts and enables the quantitative detection of a (low copy) target DNA among a larger amount of (contaminating) DNA which may be present in a single biological sample. The partitions containing the target nucleotide sequence are amplified and produce a positive detection signal, while the partitions containing no target nucleotide sequence are not amplified and produce no detection signal. However, the large number of data points retrieved from a highly sensitive assay such as dPCR is challenging to analyze. Often, it is difficult to clearly discriminate between positive and negative signals as these may overlap. Particularly, in case the biological assay did not work perfectly, e.g., the annealing of primers in the PCR assay did not work as expected, a dataset with (largely) overlapping positive and negative signals may be present. Thus, it is crucial to define the right threshold to separate real positives from real negative signals to avoid false positive and/or false negative signals. For blood screening applications, i.e., detecting Hepatitis C Virus (HCV), high sensitivity is often regarded as key feature, i.e., the proportion of positives that are correctly identified as such (true positive rate). For other assays, e.g., the detection of a mutation in a cancer-related gene such as BRCA1, the proportion of negatives that are correctly identified as such (true negative rate) may be more important.

It would be very beneficial if a predefined expectation regarding the relation of false positives to false negatives could be taken into account while a dataset from an assay is analyzed and an optimized threshold between positives and negatives is calculated automatically with regard to said predefined expectation. At the moment, no such method exists.

Currently, only simple threshold calculations, i.e., the discrimination of signals into positives and negatives based on an underlying assumption that the calls, e.g., the fluorescent signals of the dPCR partitions, are normally distributed, are available. The so calculated threshold may be manually adapted, e.g., by visual examination of the data, and a new threshold recalculated.

WO2014/153369 A1 discloses methods and systems for analyzing biological reaction systems. In particular, a method of chemically treating a surface of a substrate used in a biological reaction system to prevent biological molecules from adhering to the surface is disclosed. Further, the document relates to determining of fluorescence emission at certain locations within an image based on pixel intensity. Briefly, after determining the reaction site locations within the image, a determination of whether there is a fluorescent emission from a reaction site (positive call) or an absence of fluorescent emissions from a reaction site (negative call) can be determined based on intensities of the pixels. This may be accomplished by Otsu thresholding and various levels of thresholding may be used to discriminate between positives and negatives. Otsu thresholding, also referred to as Otsu's method, is an image processing method and is used to automatically perform clustering-based image thresholding or the reduction of a gray level image to a binary image. The algorithm assumes that the image contains two classes of pixels following bi-modal histogram (foreground pixels and background pixels), it then calculates the optimum threshold separating the two classes so that their combined spread (intra-class variance) is minimal, or equivalently (because the sum of pairwise squared distances is constant), so that their inter- class variance is maximal.

WO2014/210559 A1 discloses methods and systems for visualizing data quality including receiving a plurality of data points related to fluorescent emissions values from a plurality of reaction sites. The fluorescent emission values include information for a first type of dye and a second type of dye. For example, from a first probe labeled with FAM (6-carboxyfluorescein) and a second probe labeled with VIC (4,7,2′-trichloro-7′-phenyl-6-carboxyfluorescein) as applied in PCR assays. Different dyes associated with the data may be displayed and positive and negative calls may be determined by a processing system. However, the assumption for all of these calls, i.e., positive for VIC, positive for FAM, positive for VIC and FAM, and negative calls, is that they should be uniformly distributed. However, in real life these calls are often clumped in certain areas rather than uniformly distributed. The user is provided with a tool to visualize the types of calls and to manually adjust the threshold on a well-by-well basis or across an entire plate so that the processor will then recalculate the results based on the manually adjusted threshold. However, this procedure is very time-consuming and does not take into account a pre-defined sensitivity and specificity expectation of a certain assay.

Moreover, various methods of threshold calculation as well as respective software packages for different biological assays and especially for the analysis of dPCR data are commercially available.

However, the current methods that allow discrimination of signals into positives and negatives are based on an underlying assumption that the calls, i.e., the fluorescent signals of the dPCR droplets, are normally distributed. For example, QuantaSoft™ from Bio-Rad Laboratories, Inc. for analysis of digital PCR data (derived e.g., from the QX200™ Droplet Digital™ system) will automatically calculate a threshold above which droplets are considered positive, based on the assumption that all of the calls are uniformly distributed.

Trypsteen et al., 2015 (Trypsteen W. et al., ddpcRquant: threshold determination for single channel droplet digital PCR experiments. Analytical and bioanalytical chemistry 407.19 (2015): 5827-5834) discloses a method of threshold calculation that does not make any assumptions about the distribution of the fluorescence readouts. Briefly, a threshold is estimated by modelling the extreme values in the negative droplet population using extreme value theory and taking shifts in baseline fluorescence between samples into account.

However, none of the available data analysis methods accounts for different sensitivity and/or specificity expectations and provides a fully automated solution for research and/or diagnostic applications. Moreover, the problems of unstable thresholding for overlapping distributions of positive and negative signals and inadequate treatment of unequal effects of false-positives and false-negatives are not addressed by the current methods and/or devices.

### SUMMARY OF THE DISCLOSURE

The present disclosure is concerned with the provision of an improved method and device for analyzing a data set to allocate measurement data from a test measurement sample into either one of two different data categories. This problem is solved by the embodiments characterized in the claims and described herein below.

Therefore, the disclosure provides a method for analyzing a dataset comprising the steps of: providing a dataset comprising a plurality of measurement data from a plurality of measurement samples; providing a predefined discrimination value for separating the measurement data into two different data categories; separating the plurality of measurement data into either one of the two different data categories by determining whether the individual values of the measurement data are above or below the predefined discrimination value; determining the cumulative probability function for all values in the data category above the discrimination value (Group A) and determining the cumulative probability function for all values in the data category below the discrimination value (Group B); optimizing the ratio of the two cumulative probability functions determined in step d) with regard to a predefined error factor, thereby obtaining a new discrimination value; iterating steps c) to e); whereby the new discrimination value obtained in step e) after an iteration replaces the discrimination value of the previous iteration; and whereby the iteration is carried out until the compositions of data categories (Group A and Group B) remain constant or for a predetermined number of iterations, and g) providing the new discrimination value obtained in step f) of the last iteration as threshold for allocating measurement data from a test measurement sample into either data category.

Also provided is a device for carrying out the method disclosed herein comprising: a) a data storage unit comprising a plurality of measurement data from a plurality of measurement samples; and b) a data processing unit having tangibly embedded a computer program code carrying out the method disclosed herein.

### BRIEF DESCRIPTION OF THE FIGURES

FIGS. 1A-1B show an example for automated Positive/Negative Calling. The settings were as follows: Settings: Initial Guess: 0.8*Min+0.2 Max: 1261; Probability bias=10 (less false positives); Threshold after 8 iterations: 709.

FIGS. 2A-2B show a further example for automated Positive/Negative Calling: The settings were as follows: Initial Guess: 0.8*Min+0.2 Max: 1824; Probability bias=10 (less false positives). Threshold after 10 iterations: 1846.

FIG. 3 shows a schematic presentation of a method for analyzing a dataset according to the present disclosure.

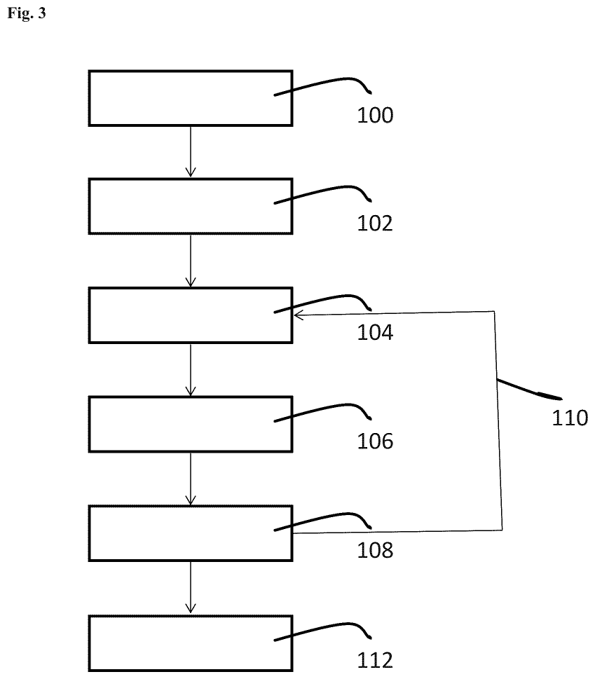

### DETAILED DESCRIPTION

As used in the following specification and claims, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used herein, the terms “particularly,” “more particularly,” “specifically,” “more specifically,” “typically,” and “more typically” or similar terms are used in conjunction with additional or alternative features, without restricting alternative possibilities. Thus, features introduced by these terms are additional or alternative features and are not intended to restrict the scope of the claims in any way. The disclosure may, as the skilled person will recognize, be performed by using alternative features. Similarly, features introduced by “in an embodiment of the disclosure” or similar expressions are intended to be additional or alternative features, without any restriction regarding alternative embodiments of the disclosure, without any restrictions regarding the scope of the disclosure and without any restriction regarding the possibility of combining the features introduced in such way with other additional/alternative or non-additional/alternative features of the disclosure.

The present disclosure relates to a method for analyzing a dataset comprising the steps of:
a) providing a dataset comprising a plurality of measurement data from a plurality of measurement samples;
b) providing a predefined discrimination value for separating the measurement data into two different data categories;
c) separating the plurality of measurement data into either one of the two different data categories by determining whether the individual values of the measurement data are above or below the predefined discrimination value;
d) determining the cumulative probability function for all values in the data category above the discrimination value (Group A) and determining the cumulative probability function for all values in the data category below the discrimination value (Group B);
e) optimizing the ratio of the two cumulative probability functions determined in step d) with regard to a predefined error factor, thereby obtaining a new discrimination value
f) iterating steps c) to e)
   whereby the new discrimination value obtained in step e) after an iteration replaces the discrimination value of the previous iteration; and
   whereby the iteration is carried out until the compositions of data categories (Group A and Group B) remain constant or for a predetermined number of iterations; and
g) providing the new discrimination value obtained in step f) of the last iteration as threshold for allocating measurement data from a test measurement sample into either data category.

The term “dataset” as used herein refers to a collection of data comprising a plurality of measurement data derived from a plurality of measurement samples. Typically, the dataset is derived from a biological assay, more typically from a biological assay that allows absolute quantification. For example, said dataset may be a dataset retrieved by performing a digital PCR (dPCR), particularly a droplet digital PCT (ddPCR). ddPCR is a method for performing digital PCR that is based on water-oil emulsion droplet technology. Here, a single test sample, for example a biological sample comprising nucleic acids, is fractionated into about 20,000 droplets, and PCR amplification of the template molecules occurs in each individual droplet. ddPCR technology uses reagents and workflows similar to those used for most standard quantitative PCR techniques, e.g., TaqMan™ probe-based assays. Thus, said plurality of measurement samples may relate to said dPCR droplets, where each droplet emits a strong, low or no fluorescent signal, resulting in a plurality of measurement data (i.e., many data points relating to fluorescence intensity values). The dataset retrieved from an analyzing assay, such as a dPCR assay, may also be pre-processed, for example the dataset to be used in the methods of the present disclosure may only contain part of the original data derived from an analyzing assay. Particularly, said plurality of measurement data relates to more than 100, more than 1,000, more than 2,000, more than 5,000, more than 10,000, more than 20,000 or more than 50,000 data points.

The measurement data may be any kind of data such as raw data, number of counts, intensity values or other data which can be derived from the measurement sample and which are indicative of at least one chemical or physical property of said measurement sample and, in particular, its ingredients. Typically, said measurement data are intensity values which can be correlated quantitatively to the amount of at least one component comprised in the measurement sample, e.g., a biomolecule such as a nucleic acid or an organism such as a cell present in the measurement sample. More typically, said intensity values are intensity values of fluorescence, chemiluminescence, radiation or colorimetric changes. For example, intensity values of fluorescence may be emitted during a nucleic acid hybridization assay or Flow Cytometry. Chemiluminescence, radiation or colorimetric changes may be detected in immunohistology and imaging assays.

More typically, said measurement data are intensity values derived from a digital PCR assay. As also explained elsewhere herein, a plurality of measurement data (e.g., fluorescence intensity data) is usually derived from a plurality of measurement samples (e.g., ddPCR droplets) derived from a single test sample (e.g., a biological sample comprising nucleic acids).

The term “test sample” as used herein refers to any type of sample from any source to be analyzed by the method of the present disclosure. Typically, the test sample is a liquid sample that can be partitioned into measurement samples. Moreover, the sample can be either an artificial sample, i.e., a sample which is artificially composed or results from an artificially initiated chemical reaction, or can be a naturally occurring sample or a sample derived from a natural source, e.g., a biological, food or environmental sample. Typically, said test sample is a biological sample. Biological samples include samples derived from an organism, typically a mammalian organism, such as body fluid samples, e.g., blood, or tissue samples, samples containing bacterial and/or viral particles as well as food samples such as fruit extracts, meat or cheese. Typical test samples include liquid samples such as samples of body fluids like blood, plasma, serum or urine, fruit juices and solid samples such as tissue samples or food extracts. Also typically, said test sample comprises nucleic acids and/or cells. It is known to those skilled in the art that a test sample, in particular, a biological test sample comprising nucleic acids and/or cells, may have been pre-processed, for example, by isolating cells or DNA from a tissue or DNA from cells. Means and methods for isolation and/or treatment of biological samples are well known in the art and include, for example, the separation of blood samples by centrifugation, the use of coated magnetic microbeads or the use of solid phase binding materials for rapidly isolating nucleic acids from samples. Typically, the test sample is a single test sample from which the plurality of measurement samples can be obtained. For example, said single test sample may be a biological sample, such as a blood sample comprising nucleic acids that is subsequently analyzed by ddPCR as explained elsewhere herein in detail, i.e., a single sample comprising nucleic acids is partitioned into around 20,000 droplets which correspond to said plurality of measurement samples and the florescence intensity of these partitions is recorded thereby providing a plurality of measurement data.

The term “predefined discrimination value” as used herein, also referred to as initial threshold, relates to a value that is used for separating the measurement data into two different data categories, e.g., positives and negatives. All values in the data category above the predefined discrimination value are considered as, e.g., positives (Group A) and all values in the data category below the predefined discrimination value are considered as, e.g., negatives (Group B). The predefined discrimination value (initial threshold) may be any value within the minimum and the maximum value of the dataset. Typically, said predefined discrimination value is the median, arithmetic mean or any percentile between the 15^{th} and the 85^{th} percentile of the measurement data.

Means and methods for the discrimination of data into different categories as well as the statistical analysis of a dataset, e.g., calculating mean or median values, normal distributions and cumulative probability functions, are known to the person skilled in the art can also be found in standard text books of statistics.

According to the present disclosure, the ratio between two cumulative probability functions for Group A (positives) and Group B (negatives) shall be optimized with regard to a predefined error factor. This means that the ratio between the two cumulative probability functions shall correspond to a predefined expectation concerning the presence of false positives or false negatives, i.e., correspond to the predefined error factor as explained elsewhere herein. Thereby a new discrimination value, i.e., a optimized threshold, is obtained.

In particular, obtaining a new discrimination value may, typically, comprise the following:
determining the intersection point between the cumulative probability functions of positives (Group A) and negatives (Group B);
calculating the probability of said intersection point deriving two new cumulative probability functions;
multiplying or dividing said new cumulative probability functions by the square root of the predefined error factor and calculating two approximation points using inverse cumulative probability functions; and
interpolating the probability density function ratio of the approximation points thereby obtaining a new discrimination value.

Means and methods to perform the above listed calculations are known to those skilled in the art and can also be found in standard textbooks relating to statistical analysis and probability calculation. Respective calculations can also be performed using computer programs such as Microsoft® Exel or MATLAB®. For example, the inverse cumulative distribution function specifies, for a given probability in the probability distribution of a random variable, the value at which the probability of the random variable is less than or equal to the given probability. An interpolation refers to a method of constructing new data points within the range of a discrete set of known data points. Interpolation methods include, for example, linear interpolation, logarithmic interpolation, polynomial interpolation, spline interpolation, interpolation via Gaussian processes, interpolation by rational functions and multivariate interpolation. According to the present disclosure, the interpolation, e.g., interpolation of the probability density function ratio of the approximation points, is typically done logarithmically.

The term “new discrimination value” as used herein, also referred to as new, better or optimized threshold, relates to a discrimination value that takes the predefined error factor into account as explained elsewhere herein. The new discrimination value is calculated as described above and derived after each iteration cycle. After carrying out a certain predetermined number of iterations until the compositions of the data categories, i.e., the number of data points in Group A and Group B, remain constant, the (final) new discrimination value is used for allocating measurement data from a test measurement sample into either data category. It will be understood that the iteration process can also be carried out as long as required until the compositions of the data categories, i.e., the number of data points in Group A and Group B, remain constant.

The term “error factor” as used herein refers to a predefined expectation regarding the presence of false positives and false negatives. In other words, the error factor relates to the desired quality of the result. The error a factor accounts for a pre-defined probability bias, e.g., fewer false positives are desired. According to the present disclosure, the ratio between the two cumulative probability functions of Group A (positives) and Group B (negatives) shall correspond to a predefined expectation concerning the presence of false positives or false negatives, i.e., to the predefined error factor. For example, a high error factor may relate to the ratio of false negatives to false positives while a low error factor may relate to the ratio of false positives to false negatives. As mentioned above, the definition of error factor depends on the desired sensitivity and specificity. The error factor may be chosen differently for each assay and pre-set at the beginning of the analysis of the dataset, i.e., the automatic calculation of a new discrimination factor with regard to the pre-set error factor in accordance with the present disclosure. A mentioned above, data sets derived from highly sensitive biological assays such as a digital PCR often show an overlapping signal distribution and a false positive or false negative call can have a considerable impact by increasing or decreasing the reported concentration considerably above or below a threshold indicating presence. For example, a false medical report based on a false positive signal could be issued, indicating the presence of a certain DNA/mutation which in reality is not present. Thus, the error factor may be chosen for each assay individually accounting for the expectation concerning the sensitivity and specificity of the result, i.e., fewer false positives or fewer false negatives, respectively.

Typically, the error factor is below or equal to 100, below or equal to 80, below or equal to 60, below or equal to 50, below or equal to 40, below or equal to 30, below or equal to 20 or below or equal to 10.

According to the method of the present disclosure, the square root of the error factor is typically used. As mentioned also elsewhere herein, typically, the intersection point of the cumulative probability functions of the negative tail of the positive group and the positive tail of the negative group is calculated followed by the calculation of the probability of this point deriving two new probabilities yielding two new cumulative probability functions. Then the error factor is used as a pre-set factor between false positive and false negative rate. The probabilities are then multiplied or divided, respectively, divided by the square root of the error factor and with the help of inverse cumulative probability functions two approximation points for the new positive negative threshold are calculates. The probability density function ratio of these approximation points is then interpolated, in one embodiment, logarithmically, to get a new discrimination value, i.e., a better estimate for the new positive negative threshold.

The term “iterating” or “iteration” as used herein refers to repeating defined steps of the method in a cyclic manner wherein each new iteration cycle starts with the outcome of the previous iteration cycle in order to approach a desired result. The iteration characterized by repeating certain steps, i.e., steps c) to i) of the method according to the present disclosure, will lead to a new discrimination value as described elsewhere herein. According to the present disclosure, the iteration starts with separating the plurality of measurement data into either one of the two different data categories by determining whether the individual values of the measurement data are above (Group A, positives) or below (Group B, negatives) the predefined discrimination value, in one embodiment, the median, arithmetic mean or any percentile between the 15^{th} and the 85^{th} percentile of the measurement data, as explained elsewhere herein. Then the resulting two groups of positives (Group A) and negatives (Group B) are analyzed, i.e., the ratio of the two cumulative probability functions (Group A and Group B) is optimized with regard to a predefined error factor thereby obtaining a new discrimination value as explained elsewhere herein. The iteration shall take place until the compositions of data categories (Group A and Group B) remain constant or for a predetermined number of iterations. Typically, the predetermined number of iterations is any number between 5 and 20, between 5 and 15, between 5 and 12 or between 5 and 10.

It will be understood that the method of the present disclosure may include additional steps and may be a computer-implemented method. Typically, the method of the present disclosure is an automated method, meaning that at least one step and more commonly, more than one step, is automated.

Typically, the method according to the present disclosure further comprises the step of performing an analyzing assay and retrieving the measurement data from said assay. An analyzing assay may be any assay, typically a biological assay that provides a plurality of measurement data from a plurality of measurement samples. In particular, the analyzing assay may comprise Flow Cytometry, nucleic acid hybridization or immunohistology and imaging. Means and methods to perform analyzing assays such as Flow Cytometry, nucleic acid hybridization or immunohistology and imaging are well known in the art and can also be found in standard text books of cell biology, immunology, molecular biology and/or biochemistry. More typically, the analyzing assay comprises nucleic acid amplification by polymerase chain reaction (PCR). PCR is well known in the art and describes a technique used to amplify DNA. PCR typically includes the steps of subjecting double stranded nucleic acids in a reaction mixture to reversibly denaturing conditions (“denaturing step”), e.g., by heating above the melting temperature of the double stranded nucleic acids in an effort to disrupt the hydrogen bonds between complementary bases, yielding single-stranded DNA molecules, annealing a primer to each of the single stranded nucleic acids (“annealing step”), and extending the primers by attaching mononucleotides to the ends of said primers using the sequence of the single stranded nucleic acid as a template for newly formed sequences (“extension/elongation step”). The processes of denaturation, annealing and elongation constitute of one cycle. The reaction cycle is repeated as many times as desired, for example between 10 and 100 times. The term “PCR” includes quantitative (qPCR) and qualitative PCR as well as any kind of PCR variant such as Asymmetric PCR. Allele-specific PCR, Assembly PCR, Dial-out PCR, Digital PCR (dPCR), Helicase-dependent amplification, Hot start PCR, In silico PCR, Intersequence-specific PCR (ISSR), Inverse PCR, Ligation-mediated PCR, Methylation-specific PCR (MSP), Miniprimer PCR, Multiplex ligation-dependent probe amplification (MLPA), Nanoparticle-Assisted PCR (nanoPCR), Nested PCR, Reverse Transcription PCR (RT-PCR), qRT-PCR, Solid Phase PCR, Thermal asymmetric interlaced PCR (TAIL-PCR), Touchdown PCR (Step-down PCR) and Universal Fast Walking. In some embodiments described herein, the PCR is digital PCR (dPCR).

Digital PCR can be used to directly quantify and clonally amplify nucleic acid strands including DNA, cDNA or RNA. The key difference between dPCR and traditional PCR lies in the method of measuring nucleic acids amounts, i.e., measurement of one fluorescence measurement (classical PCR) versus a plurality, e.g., thousands, of distinct fluorescence measurements (dPCR). Classical PCR carries out one reaction per single sample. Although, dPCR also carries out a single reaction within a sample, the sample is however separated into a large number of partitions and the reaction is carried out in each partition individually. For separation of a single sample, micro well plates, capillaries, oil emulsion, and arrays of miniaturized chambers with nucleic acid binding surfaces can be used. Droplet Digital PCR (ddPCR) is a method for performing digital PCR (dPCR) that is based on water-oil emulsion droplet technology. A single test sample is fractionated into droplets, typically, about 20,000 nanoliter-sized droplets, and PCR amplification of the template molecules occurs in each individual droplet. ddPCR technology uses reagents and workflows similar to those used for most standard quantitative PCR techniques, e.g., TaqMan™ probe-based assays which consist of template DNA (or RNA). Fluorescence-Quencher probes, primers, and a PCR master mix. The PCR solution is divided into smaller reactions (droplets) that are then made to run PCR individually. After multiple PCR amplification cycles, the samples are checked for the presence or absence of fluorescence (with a binary code of “0” and “1”). Typically, the fraction of fluorescing droplets is recorded by an instrument and analyzed by instrument software. Due to separation/partitioning of the sample and by assuming that that the molecule population follows the Poisson distribution, the distribution of target molecule within the sample can be approximated allowing for a quantification of the target strand in the PCR product.

It is known in the art that further methods and algorithms may be applied to a PCR dataset and that analysis of the large number of data points retrieved from dPCR may be challenging, for example due to overlapping distributions of positive and negative signals and inadequate treatment of unequal effects of false-positives and false-negatives. However, these obstacles are overcome by the method according to the present disclosure. Advantageously, the method of the present disclosure, in general, solves the problem of instable thresholding for overlapping distributions of positive and negative signals and inadequate treatment of unequal effects of false-positives and false-negatives. By employing an iterative procedure the optimal threshold level between positive and negative calls with regard to a predefined error factor can be determined, providing a fully automated solution for, e.g., research and/or diagnostic applications such as the aforementioned dPCR applications.

The present disclosure further relates to a computer-readable medium comprising computer program code that when run on a data processing device carries out the method of the present disclosure. Thus, the disclosure encompasses a computer program including computer-executable instructions for performing the method according to the present disclosure in one or more of the embodiments enclosed herein when the program is executed on a computer or computer network. Specifically, the computer program may be stored on a data carrier. Thus, specifically, one, more than one or even all of method steps a) to g) as indicated above may be performed by using a computer or a computer network, in one embodiment, using a computer program. The disclosure also encompasses and proposes a computer program product having program code means, in order to perform the method according to the present disclosure in one or more of the embodiments enclosed herein when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

In a specific embodiment, referring to the computer-implemented aspects of the disclosure, one or more of the method steps or even all of the method steps of the method according to one or more of the embodiments disclosed herein may be performed by using a computer or computer network. Thus, generally, any of the method steps including provision and/or manipulation of data, e.g., separating data or optimizing the ratio of two cumulative probability functions, may be performed by using a computer or computer network. Generally, these method steps may include any of the method steps, typically except for method steps requiring manual work, such as providing the samples and/or certain aspects of performing the actual measurements, e.g., performing an analyzing assay such as a dPCR and retrieving the measurement data from said assay.

Specifically, the present disclosure further encompasses:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

The present disclosure encompasses a device for carrying out the method according to the present disclosure comprising
a) a data storage unit comprising a plurality of measurement data from a plurality of measurement samples; and
b) a data processing unit having tangibly embedded a computer program code carrying out the method of the present disclosure.

Means and methods to record, store and process data are well known in the art. A data storage unit according to the present disclosure may be any means capable of storing data such as a hard drive, flash drive, CD-R or DVD-R. A data processing unit may be any platform that comprises a computer program code that is able to carry out the method of the present disclosure.

In one embodiment, the device for carrying out the method according to the present disclosure further comprises a measurement unit capable of obtaining the measurement data from the measurement samples. For example, the measurement unit may be a detector capable of obtaining fluorescence intensity values from the droplets of a ddPCR assay as explained elsewhere herein.

Moreover, the device may, typically, further comprise an analyzing unit capable of carrying out an analyzing assay. Typically, said analyzing assay comprises Flow Cytometry, nucleic acid hybridization or immunohistology and imaging. More typically, said analyzing assay comprises nucleic acid amplification by polymerase chain reaction (PCR), most typically said PCR is a digital PCR (dPCR) as explained elsewhere herein in detail.

The device may also, typically, comprise an output unit for providing the new discrimination value as threshold, preferably, on a graphical display. Suitable output units and graphical displays are well known in the art and include for example an output device incorporating a cathode ray tube on which both line drawings and text can be displayed. Such a graphical display may also be used in conjunction with a light pen to input or reposition data. Moreover, the output unit shall preferably, e.g., via a graphical display, further allocate additional information on said threshold.

The above explanations and definitions of the terms apply throughout the specification. Moreover, in the following, typical embodiments of the present disclosure are listed.

Embodiment 1: A method for analyzing a dataset comprising the steps of:
a) providing a dataset comprising a plurality of measurement data from a plurality of measurement samples;
b) providing a predefined discrimination value for separating the measurement data into two different data categories;
c) separating the plurality of measurement data into either one of the two different data categories by determining whether the individual values of the measurement data are above or below the predefined discrimination value;
d) determining the cumulative probability function for all values in the data category above the discrimination value (Group A) and determining the cumulative probability function for all values in the data category below the discrimination value (Group B);
e) optimizing the ratio of the two cumulative probability functions determined in step d) with regard to a predefined error factor, thereby obtaining a new discrimination value
f) iterating steps c) to e)
   whereby the new discrimination value obtained in step e) after an iteration replaces the discrimination value of the previous iteration; and
   whereby the iteration is carried out until the compositions of data categories (Group A and Group B) remain constant or for a predetermined number of iterations; and
g) providing the new discrimination value obtained in step f) of the last iteration as threshold for allocating measurement data from a test measurement sample into either data category.

Embodiment 2: The method of embodiment 1, wherein said measurement data are intensity values.

Embodiment 3: The method of embodiment 2, wherein said intensity values are intensity values of fluorescence, chemiluminescence, radiation or colorimetric changes.

Embodiment 4: The method of any one of embodiments 1 to 3, wherein said plurality of measurement samples is derived from a single test sample.

Embodiment 5: The method of embodiment 3, wherein said test sample is a biological sample.

Embodiment 6: The method of embodiment 5, wherein said biological sample comprises nucleic acids or cells.

Embodiment 7: The method of any one of embodiments 1 to 6, wherein said predefined discrimination value for separating the measurement data into two different data categories is the median, arithmetic mean or any percentile between the 15th and the 85th percentile of the measurement data.

Embodiment 8: The method of any one of embodiments 1 to 7, wherein step e) comprises:
i. determining the intersection point between the cumulative probability functions of step d);
ii. calculating the probability of said intersection point deriving two new cumulative probability functions
iii) multiplying or dividing said new cumulative probability functions by the square root of the predefined error factor and calculating two approximation points using inverse cumulative probability functions;
iv) interpolating the probability density function ratio of the approximation points thereby obtaining a new discrimination value.

Embodiment 9: The method of any one of embodiments 1 to 8, wherein the error factor is below or equal to 100, below or equal to 80, below or equal to 60, below or equal to 50, below or equal to 40, below or equal to 30, below or equal to 20 or below or equal to 10.

Embodiment 10: The method of any one of embodiments 1 to 9, wherein said predetermined number of iterations is any number between 5 and 20, between 5 and 15, between 5 and 12 or between 5 and 10.

Embodiment 11: The method of embodiment 8, wherein said interpolating is logarithmic.

Embodiment 12: The method of any one of embodiments 1 to 11, wherein said method is a computer-implemented method.

Embodiment 13: The method of any one of embodiments 1 to 12, wherein the method further comprises the step of performing an analyzing assay and retrieving the measurement data from said assay.

Embodiment 14: The method of embodiment 13, wherein said analyzing assay comprises Flow Cytometry, nucleic acid hybridization or immunohistology and imaging.

Embodiment 15: The method of embodiment 14, wherein said analyzing assay comprises nucleic acid amplification by polymerase chain reaction (PCR).

Embodiment 16: The method of embodiment 15, wherein said PCR is digital PCR (dPCR).

Embodiment 17: A computer-readable medium comprising a computer program code that when run on a data processing device carries out the method of any one of embodiments 1 to 16.

Embodiment 18: A device for carrying out the method of any one of embodiments 1 to 16 comprising:
a) a data storage unit comprising a plurality of measurement data from a plurality of measurement samples; and
b) a data processing unit having tangibly embedded a computer program code carrying out the method of any one of embodiments 1 to 16.

Embodiment 19: The device of embodiment 18, wherein said device further comprises a measurement unit capable of obtaining the measurement data from the measurement samples.

Embodiment 20: The device of embodiment 18 or 19, further comprising an analyzing unit capable of carrying out an analyzing assay.

Embodiment 21: The device of embodiment 20, wherein said analyzing assay comprises Flow Cytometry, nucleic acid hybridization or immunohistology and imaging.

Embodiment 22: The device of embodiment 20, wherein said analyzing assay comprises nucleic acid amplification by polymerase chain reaction (PCR).

Embodiment 23: The device of embodiment 22, wherein said PCR is digital PCR (dPCR).

Embodiment 24: The device of any one of embodiments 18 to 23, wherein said device further comprises an output unit for providing the new discrimination value as threshold, preferably, on a graphical display.

Embodiment 25: The device of embodiment 24, wherein said output unit further allocates additional information on the threshold.

### EXAMPLES

The following Examples shall illustrate the disclosure. They shall, by no means, construed as limiting the scope for which protection is sought.

### Example: Algorithm for Positive/Negative Calling

In a first step, an initial guess for threshold is made. Subsequently, the distribution parameter of positives and negatives is determined automatically. To this end, the threshold is set so that the derived probabilities of false-positives and false-negatives fulfill a pre-set probability ratio. An iterative application is carried out next to improve threshold level until ratio of positives and negatives remain constant. The concept here consists in performing an iterative procedure to determine the optimal threshold level between positive and negative call (compared to just use a pre-set cutoff). The iteration starts with an initial threshold, e.g., the (weighted) mean of the maximal and minimal signal level. Then the resulting two groups of positives and negatives are analyzed. Based on an adequate pre-set subset of the data e.g., Percentile range 5% at inner to 99% at outer range (for possibly overlapping distributions) the theoretical cumulative probability distribution is calculated for each group (positives and negatives), e.g., the error function for normal distributions. In a next step, the intersection point of the cumulative probability functions of the negative tail of the positive group and the positive tail of the negative group is calculated. Afterwards, the probability of this point is calculated. Subsequently, a pre-set factor between false positive and false negative rate is used. The probabilities are to be divided or multiplied with by the square root of this factor and with the help of the inverse cumulative probability functions two approximation points for the new positive negative threshold are calculated. The probability density function ratio of these points is logarithmically interpolated to get a good estimate for the new positive negative threshold. This iteration is repeated until the number of positives and negatives remains constant or at maximum a pre-set times, e.g., 10 times.

The aforementioned algorithm can be implemented by the following program instructions:
Crossing Point and (logarithmic) Interpolation Derivation
Clear[p1,p2,x,a1,a2,b1,b2];
(* simplified approximation for cumulative normal distribution probability as function of z value *)
p1=Module[{a1,b1,x,z1=(x−a1)/b},
1−2\hat{ }(−22\hat{ }(1−41\hat{ }(z1/10)))];
p2=Module[{a2,b2,x,z2=(a2−x)/b2},
1−2\hat{ }(−22\hat{ }(1−41\hat{ }(z2/10)))];
Simplify[Solve[p1==p2,x]] [[1]]
Solve:ifun: Inverse functions are being used by Solve, so some solutions may not be found; use Reduce for complete solution information.>>
{x->(b1(a2 Log[41]−10 b2 Log[41\hat{ }(−(a1/(10 b1)))]))/((b1+b2)Log[41])}
Clear[y,y1,y2,x2,x1,x];
Simplify[Solve[y==y1+(y2−y1)/(x2−x1) (x−x1),x]]
{{x->(x1 y−x2 y+x2 y1−x1 y2)/(y1−y2)}}
Auxiliary Functions ((inverse) cumulative probability density)
Clear[pr,ipr];
(* probability of cumulative normal distribution as function of z value [approximation]*)
pr[z_]=Module[{a=17,b=17 Pi/2,x},
x=If[z>0,z,0.5];
SetPrecision[1−(0.5+0.5 Sqrt[1−Exp[−x\hat{ }2(a+x\hat{ }2)/(b+2x\hat{ }2)]]), 100]];
(* inverse probability of cumulative normal distribution (z value) as function of probability [approximation]*)
ipr[y_]=Module[{a=17,b=17 Pi/2},
Sqrt[−a−2 Log[4 y−4 y\hat{ }2]+Sqrt[−4 b Log[4 y−4 y\hat{ }2]+(a+2 Log[4 y−4 y\hat{ }2)\hat{ }2]]/Sqrt[2]];
Iteration Function (data, initial cutoff and optional spread delivers new cutoff)
(* calculate new positive/negative cutoff *)
Clear[cutoffnew];
cutoffnew[datain_List,cin _?NumberQ,Optional[spec_?NumberQ,1]]:=Module[
(* variables used: *)
{data (* all data *),
cold, (* intial cutoff *)
low, high, (* limits for initial cutoff *)
dn,dp, (* negative and positive data part, truncated *)
qn,qp, (* quantile limits, 10% at inner and 1% at outer edges *)
nn,np, (* number of negatives/positives *)
mn,mp, (* median of negatives/positives *)
sn,sp, (* standard deviation of negatives/positives *)
xn,xp, (* z value of initial cutoff in negative/positive distribution *)
pn,pp, (* probability of negative/positive cumulative distribution tail *)
c0,(* intersection of cumulative probability density tails *)
f, (* square root of spreading factor *)
cn,cp, (* cutoff approximations from negative/positive cumulative distribution tail *)
dlpn,dlpp, (* log probability of negative/positive cumulative distribution tail from approximations *)
cnew (* new resulting cutoff *)},
(* Filter out non-numerical data and use default starting cutoff in case input is out of range *)
data=Select[datain, NumberQ]; low=Quantile[data,0.01]; high=Quantile[data,0.99]; cold=If[Or[cin<low,cin>high],0.8low+0.2high,cin];
(* calculate features of initial negatives *)
dn=Select[data,#<=cold&]; qn=Quantile[dn, {0.01,0.95}]; dn=Select[dn,And[#>=qn[[1]], #<=qn[[2]]]&];
nn=Length[dn]; mn=Median[dn]//N; sn=StandardDeviation[dn]//N;
xn=(c0−mn)/sn;
(* calculate features of initial positives *)
dp=Select [data,#>cold&]; qp=Quantile[dp, {0.05,0.99}]; dp=Select[dp, And[#>=qp[1]], #<=qp[[2]]]&];
np=Length[dp]; mp=Median[dp]/N; sp=StandardDeviation[dp]//N;
xp=(mp−c0)/sp;
(* calculate intersection of cumulative probability density tail functions of negatives and positives *)
c0=(mp−10 sp Log[41\hat{ }(−mn/(10 sn))]/Log[41])/(1+sp/sn);
f=Sqrt[spec(nn/np)];
(* spread (by factor f\hat{ }2) cumulative probability density functions of negatives and positives *)
cn=Max[mn,Min[mp,mn+sn ipr[1/f sn/sp pr[SetPrecision[(c0−mn)/sn, 100]]]]];
cp=Max[mn,Min[mp,mp−sp ipr[1/(2+1/(f sn/sp pr[SetPrecision[(mp−c0)/sp, 100]]))]]];
dlpn=Log[pr[SetPrecision[(mp−cn)/sp, 100]]]−Log[pr[SetPrecision[(cn−mn)/sn, 100]]];
dlpp=Log[pr[SetPrecision[(mp−cp)/sp, 100]]]−Log[pr[SetPrecision[(cp−mn)/sn, 100]]];
(* calculate new cutoff as interpolation *)
cnew=((cn−cp)Log[f\hat{ }2]+cp dlpn−cn dlpp)/(dlpn−dlpp);
(* total result output *)
{cnew,c0,cn,cp,nn,np,(c0−mn)/sn,(mp−c0)/sp,
pr[SetPrecision[(cnew−mn)/sn,100]]//N,pr[SetPrecision[(mp−cnew)/sp,100]]//N,
pr[SetPrecision[(mp−cnew)/sp,100]]/pr[SetPrecision[(cnew−mn)/sn,100]]//N,f\hat{ }2/N}]

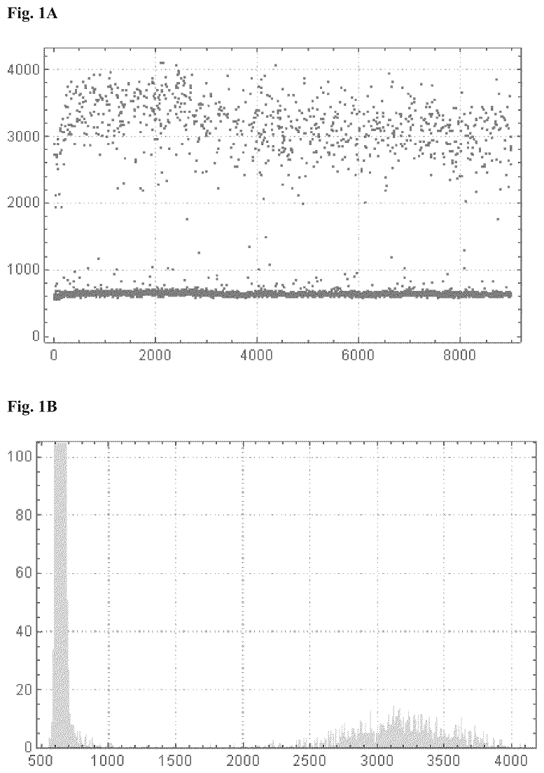

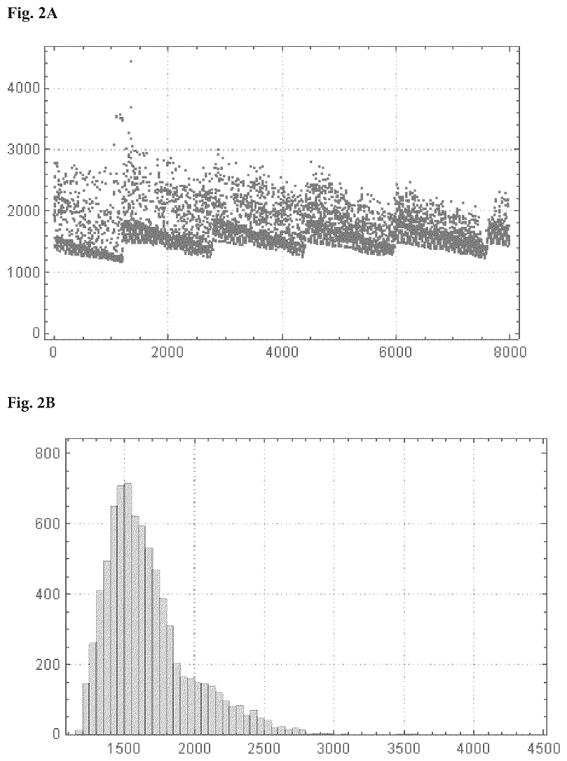

Using this program to implement the algorithm, data could be analyzed as shown in FIGS. 1 and 2. The settings in the experiment analyzed in FIG. 1 were as follows: Settings: Initial Guess: 0.8*Min+0.2 Max: 1261; Probability bias=10 (less false positives); Threshold after 8 iterations: 709. The settings in the experiment analyzed in FIG. 2 were as follows: Initial Guess: 0.8*Min+0.2 Max: 1824; Probability bias=10 (less false positives). Threshold after 10 iterations: 1846.

The present application is not to be limited in scope by the specific embodiments described herein. Indeed, various modifications in addition to those described herein will become apparent to those skilled in the art from the foregoing description and accompanying figures. Such modifications are intended to fall within the scope of the claims. Various publications are cited herein, the disclosures of which are incorporated by reference in their entireties.

### LIST OF REFERENCE NUMBERS

100: provision of a dataset comprising a plurality of measurement data from a plurality of measurement samples
102: provision of a predefined discrimination value for separating the measurement data into two different data categories
104: separation of the plurality of measurement data into either one of the two different data categories by determining whether the individual values of the measurement data are above or below the predefined discrimination value
106: Determination of the cumulative probability function for all values in the data category above the discrimination value (Group A) and determining the cumulative probability function for all values in the data category below the discrimination value (Group B)
108: Optimizing the ratio of the two cumulative probability functions determined in step d) with regard to a predefined error factor, thereby obtaining a new discrimination value
110: Iteration of steps 104 to 108 whereby the new discrimination value obtained in step e) after an iteration replaces the discrimination value of the previous iteration and whereby the iteration is carried out until the compositions of data categories (Group A and Group B) remain constant or for a predetermined number of iterations
112: Provision of the new discrimination value obtained in step 110 of the last iteration as threshold for allocating measurement data from a test measurement sample into either data category

1-13. (canceled)

14. A computer-implemented method for analyzing a dataset, the method comprising a step of performing an analyzing assay and retrieving intensity values from said assay, wherein said analyzing assay comprises nucleic acid amplification by digital polymerase chain reaction (dPCR),
wherein, in the digital PCR, the sample is separated into partitions, wherein a PCR reaction is carried out in each partition individually, wherein partitions containing a target nucleotide sequence are amplified and produce a positive detection signal, while the partitions containing no target nucleotide sequence are not amplified and produce no detection signal;
further comprising the steps of:
a) providing a dataset comprising a plurality of intensity values from a plurality of measurement samples; wherein said plurality of measurement samples are derived from a single biological test sample comprising nucleic acids;
b) providing a predefined discrimination value for separating the intensity values into two different data categories;
c) separating the plurality of intensity values into either one of the two different data categories by determining whether the individual values of the intensity values are above or below the predefined discrimination value;
d) determining the cumulative probability function for all values in the data category above the discrimination value (Group A) and determining the cumulative probability function for all values in the data category below the discrimination value (Group B);
e) obtaining a new discrimination values such that a ratio between the two cumulative probability functions corresponds to a predefined error factor;
f) iterating steps c) to e)
whereby the new discrimination value obtained in step e) after an iteration replaces the discrimination value of the previous iteration; and
whereby the iteration is carried out until the compositions of data categories (Group A, positives and Group B, negatives) remain constant or for a predetermined number of iterations; and
g) automatically providing the new discrimination value obtained in step f) of the last iteration as threshold for allocating intensity values derived from a digital PCR assay from the biological sample comprising nucleic acids into either data category, Group A positives and Group B negative.

15. The method of claim 1, wherein said intensity values are intensity values of fluorescence, chemiluminescence, radiation or colorimetric changes.

16. The method of claim 1, wherein said predefined discrimination value for separating the intensity values into two different data categories is the median, arithmetic mean or any percentile between the 15^{th} and the 85^{th} percentile of the intensity values.

17. The method of claim 1, wherein step e) comprises:
i. determining the intersection point between the cumulative probability functions of step d);
ii. calculating the probability of said intersection point deriving two new cumulative probability functions
iii) multiplying or dividing said new cumulative probability functions by the square root of the predefined error factor and calculating two approximation points using inverse cumulative probability functions;
v) interpolating the probability density function ratio of the approximation points thereby obtaining a new discrimination value.

18. The method of claim 1, wherein the error factor is below or equal to 100, below or equal to 80, below or equal to 60, below or equal to 50, below or equal to 40, below or equal to 30, below or equal to 20 or below or equal to 10.

19. The method of claim 1, wherein said predetermined number of iterations is any number between 5 and 20, between 5 and 15, between 5 and 12 or between 5 and 10.

20. The method of claim 1, further comprising the following step:
h) obtaining a constant number of data points in Group A and Group B.

21. The method of claim 1, wherein the error factor is below or equal to 10.

22. The method of claim 1, wherein the error factor is a predefined expectation regarding the presence of false positives and false negatives.

23. The method of claim 1, further comprising the following step: i) allocating the measurement data into either one of the two different data categories.

24. The method of claim 1, wherein the dataset is a dataset retrieved by performing droplet digital PCR (ddPCR).

25. A computer-readable medium comprising a computer program code that when run on a data processing device carries out the method of claim 1.

26. A device for carrying out the method of claim 1 comprising:
a) a data storage unit comprising a plurality of intensity values from a plurality of measurement samples; and
b) a data processing unit having tangibly embedded a computer program code carrying out the method of claim 1.

27. The device of claim 26, wherein said device further comprises a measurement unit capable of obtaining the intensity values from the measurement samples and, preferably, an analyzing unit capable of carrying out an analyzing assay.
